(19) United States
(12) Patent Application Publication
(10) Pub. No.: US 2006/0173472 A1
(43) Pub. Date: Aug. 3, 2006

(54) Gastric banding device

(76) Inventors: Warren L. Starkebaum, Plymouth, MN (US); Yelena G. Tropsha, Plymouth, MN (US)

Correspondence Address: SHUMAKER & SIEFFERT, P. A., 8425 SEASONS PARKWAY, SUITE 105, ST. PAUL, MN, 55125, US

(21) Appl. No.: 11/047,376

(22) Filed: Jan. 31, 2005

(51) Int. Cl. A61B 17/08

(52) U.S. Cl. 606/153

(57) ABSTRACT

A gastric banding device contains an expandable material. The expandable material may include, for example, a hydrogel material that expands when hydrated. The hydrogel material may include a single or multiple monolithic components, and/or may include hydrogel material in particulate form, such as hydrogel beads, microspheres, or powder. The gastric banding device includes an elongate gastric band having an expandable lumen that forms a stoma opening in the stomach by encircling and partitioning the stomach into an upper stomach and a lower stomach. The hydrogel material is positioned in the lumen and expands when hydrated to at least partially expand the lumen, thus decreasing the size of the stoma opening.

### FIELD OF THE INVENTION

The invention relates to medical devices and methods, and in particular, to devices for the treatment of obesity.

### BACKGROUND

Various surgical techniques have been developed to treat morbid obesity. One of these techniques involves use of a gastric banding device. Conventional hydraulic gastric bands are typically constructed in the form of a hollow tube fabricated from an elastomer, such as silicone rubber. The band can be inserted through a laproscopic cannula to completely encircle the upper end of the stomach and thus restrict the passage of food into the lower stomach. Saline is injected or withdrawn from the band by inserting a needle into an injection port placed just under the patient's skin. The degree of gastric constriction provided by the band, which affects the amount of food the patient can ingest, can be adjusted by varying the amount of saline in the band.

These conventional hydraulic gastric banding devices exert a continuous restricting force on the stomach to reduce the size of the upper stomach and to restrict the passage of food from the upper to the lower stomach. However, because saline passively diffuses through the walls of the elastomer, hydraulic bands do not offer stable banding over time. Additional medical procedures may therefore be necessary to refill the band to maintain its optimal configuration, increasing the cost and the number of medical visits. Also, each time the band is refilled, the patient's skin must be punctured, resulting in discomfort for the patient and an increased risk of infection.

### SUMMARY

In general, the invention is directed to a gastric banding device containing an expandable material. The expandable material may include, for example, a hydrogel material that expands when hydrated. The hydrogel material may include a single or multiple monolithic components, and/or may include hydrogel material in particulate form, such as hydrogel beads, microspheres, or powder. The gastric banding device includes an elongate gastric band having an expandable lumen that forms a stoma opening in the stomach by encircling and partitioning the stomach into an upper stomach and a lower stomach. A hydrogel material is positioned in the lumen and expands when hydrated to at least partially expand the lumen, thus decreasing the size of the stoma opening. After a period of time, the hydrogel material reaches equilibrium. Because hydrogel materials are very stable at equilibrium, the problem of fluid leakage experienced by conventional gastric banding systems is reduced and the geometric configuration of the band may be maintained for long periods of time. The band may also be used with other portions of the gastrointestinal (GI) tract, such as the esophagus, intestines, etc.

In one embodiment, the invention is directed to a device comprising an elongate gastric band having a radially expandable lumen that forms a stoma opening in the stomach by encircling and partitioning a stomach into an upper stomach and a lower stomach, and a hydrogel material positioned in the lumen that expands when hydrated to at least partially expand the lumen.

In another embodiment, the invention is directed to a device comprising a gastric occluding device positioned to restrict ingestion of food by a patient and having at least one expandable lumen, and an expandable material positioned within the lumen that expands when hydrated to at least partially expand the lumen.

In another embodiment, the invention is directed to method comprising implanting a gastric band having at least one expandable lumen with material that expands when hydrated positioned therein such that the gastric band forms a stoma opening in the stomach of a patient by encircling and partitioning a stomach into an upper stomach and a lower stomach, and injecting fluid into the expandable lumen to at least partially hydrate the material and to decrease the stoma opening.

In another embodiment, the invention is directed to a method comprising implanting a gastric band having at least one expandable lumen filled with a fully hydrated hydrogel material such that the gastric band forms a stoma opening in the stomach of a patient by encircling and partitioning a stomach into an upper stomach and a lower stomach.

In another embodiment, the invention is directed to a method comprising implanting a gastric band having at least one expandable lumen such that the gastric band forms a stoma opening in the stomach of a patient by encircling and partitioning a stomach into an upper stomach and a lower stomach, and injecting a slurry of particulate hydrogel material into the expandable lumen to at least partially expand the lumen and decrease the stoma opening.

In another embodiment, the invention is directed to a device comprising an elongate gastric band having a radially expandable lumen that forms a stoma opening in the stomach by encircling and partitioning a stomach into an upper stomach and a lower stomach, and an injection port in fluid connection with the lumen to receive and deliver a slurry of particulate hydrogel material into the expandable lumen to at least partially expand the lumen and decrease the stoma opening.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a diagram illustrating an example gastric banding device positioned around a stomach of a patient.

FIG. 2 is a lengthwise cross-sectional view of the gastric banding device of FIG. 1.

FIG. 3 is a cross sectional side view of the gastric banding device of FIG. 1 taken along line A-A.

FIG. 4A is a cross-sectional view of the gastric banding device taken along the line B-B in FIG. 2 having dehydrated or partially hydrated hydrogel material within a lumen of the band.

FIG. 4B is a cross-sectional view of the gastric banding device taken along the line B-B in FIG. 3 having a hydrogel material at equilibrium within a lumen of the band.

FIG. 5 is a lengthwise cross-sectional view of a gastric banding device filled with a single monolithic component of hydrogel material.

FIG. 6 is a lengthwise cross-sectional view of a gastric banding device filled with a particulate hydrogel material.

FIG. 7 is a flowchart illustrating a method of implanting and hydrating a hydrogel filled gastric banding device.

FIG. 8 is a flowchart illustrating a method of implanting a gastric banding device and injecting a slurry of particulate hydrogel material.

### DETAILED DESCRIPTION

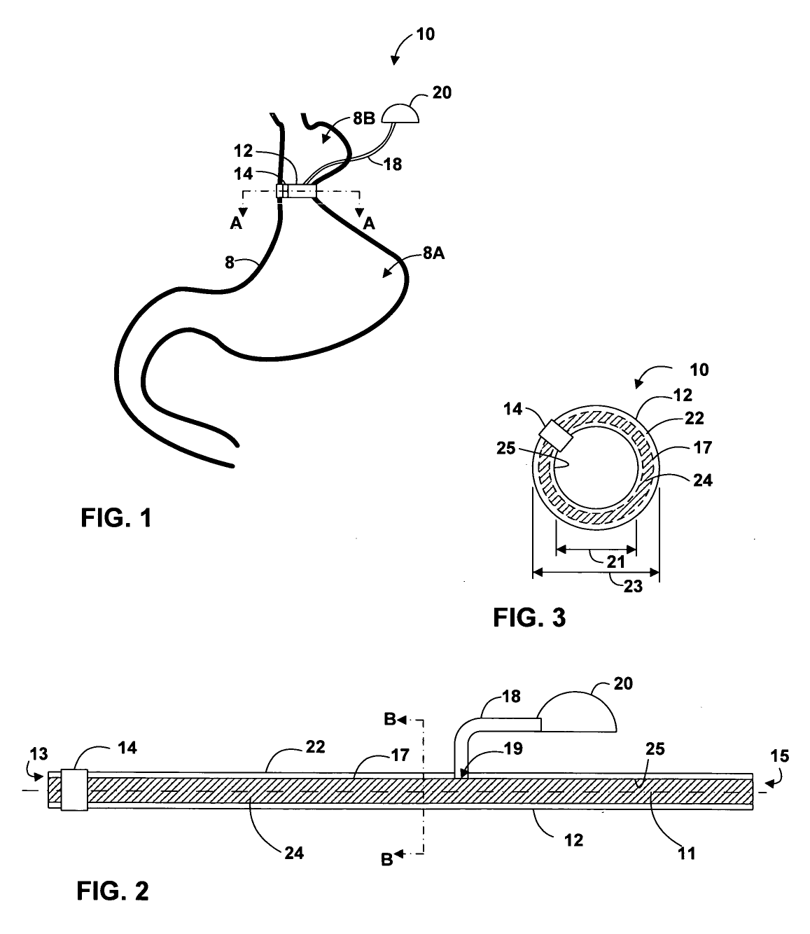

FIG. 1 is a diagram illustrating an example gastric banding device 10 positioned around a stomach 8 of a patient. Once implanted in the patient, band 12 forms a stoma opening in the stomach 8 by encircling and partitioning a stomach 8 into an upper stomach 8A and a lower stomach 8B. The degree of gastric constriction provided by band 12 (and thus the size of the stoma opening) is designed to limit the ingestion of food and reduce caloric intake so that the patient loses weight while permitting the ingestion of water and the minimum amount of caloric energy necessary to prevent malnourishment. Although in FIG. 1 band 12 is shown positioned around the top end (fundus) of the stomach 8 in a position commonly associated with an adjustable gastric banding (AGB) procedure, the band may also be placed vertically, as for a vertical banded gastroplasty (VBG), or in any other positioned designed to reduce food intake. The band may also be used with other portions of the gastrointestinal (GI) tract, such as the esophagus, intestines, etc.

FIG. 2 is a lengthwise cross-sectional view of gastric banding device 10 of FIG. 1 and FIG. 3 is a cross sectional view of the gastric banding device of FIG. 1 taken along line A-A. Band 12 of gastric banding device 10 includes an expandable lumen 17 extending longitudinally from a first end 13 to a second end 15 of band 12. In use, lumen 17 is at least partially filled with an expandable material 24. Expandable material 24 may include, for example, a hydrogel material that swells upon exposure to moisture. When exposed to moisture in its dehydrated or partially hydrated state, hydrogel material 24 absorbs water and exhibits swelling behavior. Swelling of hydrogel material 24 causes lumen 17 within band 12 to expand and the inside diameter 21 of band 12 to decrease. After the hydrogel material 24 reaches its equilibrium state, a stable band geometry may be maintained. Because hydrogel materials are very stable at equilibrium, the problem of fluid leakage and the concomitant degradation of the optimal band configuration experienced by conventional gastric banding systems may therefore be reduced or eliminated. An optimal geometric configuration of the band may be maintained for long periods of time.

Hydrogels are networked structures of polymer chains that are crosslinked to each other. In the presence of an aqueous solution, such as water, saline, body fluids, etc., the polymer chains absorb water and swell. Hydrogels can therefore assume a dehydrated state, a partially hydrated state, and a fully hydrated, or equilibrium, state. A hydrogel material in its dehydrated state is generally substantially smaller than the material in its hydrated state. Examples of hydrogel materials which may be used are the polyacrylonitrile copolymers as described in U.S. Pat. Nos. 4,943,618 and 5,252,692, which are incorporated herein by reference. Other types of hydrogel materials may include a co-polymer of polyacrylonitrile and polyacrylamide. Other hydrogel materials, however, could also be used. The hydrogel materials can be crosslinked or non-crosslinked, they can belong to the class of homopolymers or copolymers or the blends of these materials with each other. Other examples of synthetic hydrophilic polymers that can be used include, but are not limited to, polyacrylic acid, polymethacrilic acid, polyacrylaamid, polyhydroxyethylmethacrylate, polyhydroxyetrhyl methacrylate, polyvinylalcohol, polyethylene oxide, polyvinylpyrrolidone, polyuerethane, polysiloxanes, or poluethylenimines.

By controlling relative amounts of copolymers, it may be possible to regulate physical qualities of the hydrogel such as flexibility of the hydrogel material at equilibrium, the degree of swelling, and/or the rate of swelling. Depending upon the composition of hydrogel material 24 and the amount of fluid, it may take hydrogel material 24 from a few minutes to a few hours to expand and reach equilibrium. Depending upon its composition, hydrogel material 24 may absorb an amount of fluid such that its weight percent of water in the equilibrium state is anywhere between approximately 10% and 99%. The hydrogel will swell as a function of the amount of water absorbed and the composition of the hydrogel material. The physical properties of the hydrogel material may be chosen based on, among other things, the needs of each specific patient and the desired size of the stoma opening.

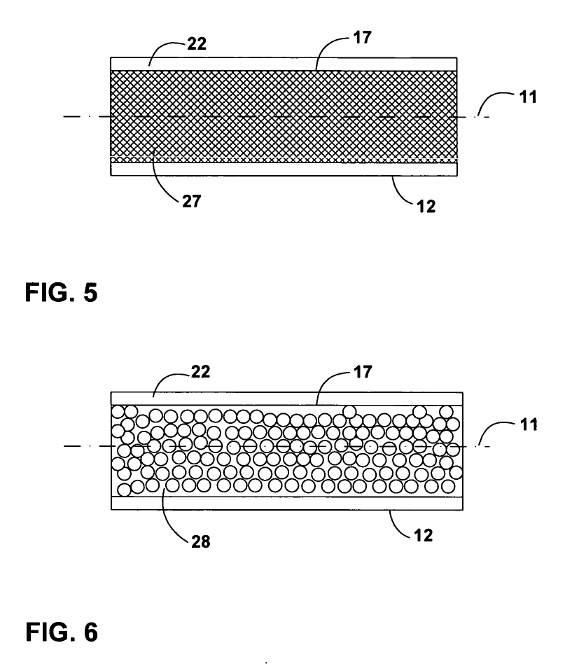

Hydrogel material 24 may include a single or multiple monolithic components, and/or may include hydrogel material in particulate form, such as hydrogel beads, microspheres, or powder. FIG. 5, for example, is a partial lengthwise cross-sectional view of a portion of gastric banding device 10 filled with a single monolithic component of hydrogel material 27. FIG. 6, for example, is a partial lengthwise cross-sectional view of a gastric banding device 10 filled with a particulate hydrogel material 28.

In one embodiment, hydrogel material 24 is fully hydrated within lumen 17 before the band 12 is implanted within the patient. In this embodiment, the physician ses the band as it will be once implanted in the patient. In another embodiment, hydrogel material 24 is positioned within lumen 17 in a dehydrated or partially hydrated state at the time of implantation. Fluid is delivered to lumen 17 after implantation to at least partially hydrate the hydrogel material, causing the band 12 to expand and decrease the size of the stoma opening. In another embodiment, a slurry of fluid and particulate hydrogel material is delivered to the lumen within the band 12 via an injection port. Once inside band 12, the particulate hydrogel material absorbs the fluid and swells, causing the band 12 to expand and decrease the size of the stoma opening. In any embodiment, additional fluid may be delivered to further hydrate/expand band 12 and thus adjust the size of the stoma opening. In another embodiment, after band 12 is placed and secured around the stomach, water will diffuse through the elastomer tube and be absorbed by hydrogel material 24, causing the band to swell, and the inside diameter 21 of the band to decrease.

To implant band 12 within a patient, a physician positions band 12 around the stomach 8 until the desired diameter of band 12 is reached (see FIG. 3). Band 12 has an inside diameter 21 and an outside diameter 23. The inside diameter 21 of band 12 determines the size of the stoma opening in the stomach. Once the desired inside diameter 21 of band 12 is formed, the two ends of band 12 are connected together via connection mechanism 14. Connection mechanism 14 may be any type of fastening mechanism adapted to attach the two ends of band 12 together. Connection mechanism 14 may include, for example, a buckle, sutures, clamp, adhesive, surgical staples, coupling, or any other type of biocompatible fastener.

FIGS. 1 and 2 also show a subcutaneous injection port 20 in fluid communication with the lumen 17 via tubing 18 and aperture 19 in a side wall 25 of lumen 17. A hydrating fluid, such as saline, may be injected into lumen 17 of band 12 to hydrate hydrogel material 24 by inserting a needle into injection port 20, which may be placed just under the patient's skin. In another embodiment, the dimensions of hydrogel material 24 in its dehydrated state can be selected such that hydrogel material 24 can fit inside the bore of an insertion device, such as needle, hollow trocar, endoscope, catheter or cannula. In this way, in some embodiments, hydrogel material 24 may be injected into lumen 17 via percutaneous fluid communication with injection port 20 after implantation of the band 12. In another embodiment, particulate hydrogel material (such as beads, microspheres, powder, etc.) may be mixed with fluid immediately before injection to assist flow of the hydrogel material through the injection needle and throughout lumen 17.

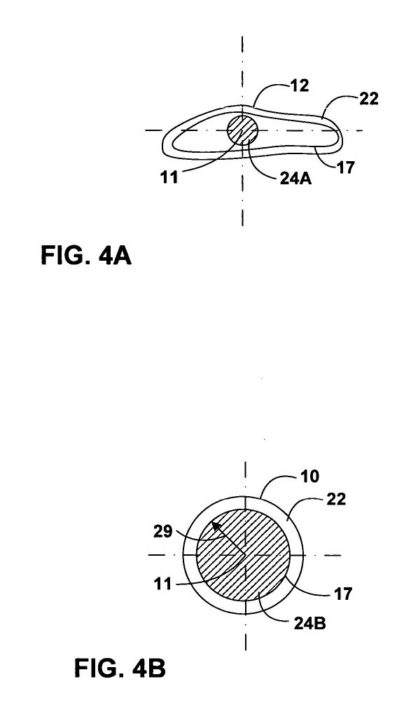

FIGS. 4A and 4B illustrate one example of how a hydrogel material 24 may expand when hydrated to at least partially expand lumen 17, thus expanding band 12. FIG. 4A is a cross-sectional view of the gastric banding device taken along the line B-B in FIG. 2. Dehydrated or partially hydrated hydrogel material 24 within a lumen of the band. FIG. 4B is a cross-sectional view of the gastric banding device taken along the line B-B in FIG. 3 showing hydrogel material 24 at equilibrium.

In the example shown in FIGS. 4A and 4B, hydrogel material 24 swells radially when hydrated. That is, hydrogel material 24 expands radially outward from axis 11 as indicated by reference numeral 29. While in a dehydrated state or a partially hydrated state, hydrogel material 24 has a small perimeter/volume as shown in FIG. 4A, but in the hydrated state as shown in FIG. 4B, hydrogel material has a larger perimeter and larger volume. As noted above, the degree of expansion can be regulated based on the degree of hydration and the type of hydrogel material used.

In embodiments where some or all of hydrogel material 24 is in particulate form, hydrogel could be removed from the device via injection port 20 using a syringe and needle if it were determined that band 12 is too tight and that the stoma opening is too small. Residual water not absorbed by hydrogel material 24 may also be removed. Conversely, band 12 may be tightened by adding additional particulate hydrogel (in either the hydrated or dehydrated form) to the band via injection port 20.

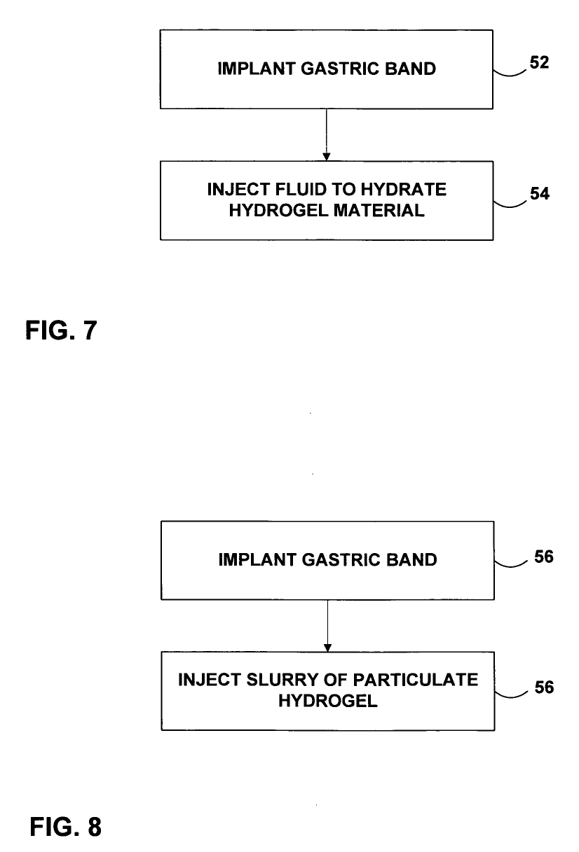

FIG. 7 is a flowchart illustrating a method of implanting and hydrating a hydrogel filled gastric banding device. In this embodiment, hydrogel material 24 is positioned within lumen 17 in a dehydrated or partially hydrated state. Band 12 is then implanted and placed into position around the patient's stomach or other appropriate portion of the GI tract (52). Fluid is delivered to lumen 17 after implantation to at least partially hydrate the hydrogel material (54), causing the band 12 to expand and decrease the size of the stoma opening.

FIG. 8 is a flowchart illustrating a method of implanting a gastric banding device and injecting a slurry of particulate hydrogel material. In this embodiment, a band 12 is implanted and placed into position around the patient's stomach or other appropriate portion of the GI tract (56). A slurry of particulate hydrogel material is delivered to the lumen within the band 12 (58). Once inside band 12, the particulate hydrogel material absorbs the fluid and swells, causing the band 12 to expand and decrease the size of the stoma opening.

In either of the embodiments shown in FIGS. 7 or 8, or in other embodiments described herein, the size of the stoma opening may be adjusted at any time by delivering additional fluid to further hydrate/expand band 12. Similarly, in some embodiments, after band 12 is placed and secured around the stomach, water will diffuse through the elastomer tube and be absorbed by hydrogel material 24, causing the band to swell, and the inside diameter of the band to decrease. The stoma opening may also be adjusted by removing residual fluid or particulate hydrogel material via injection port 20.

The preceding specific embodiments are illustrative of the practice of the invention. It is to be understood, therefore, that other expedients known to those skilled in the art or disclosed herein may be employed without departing from the invention or the scope of the appended claims. For example, the invention is not limited to the particular shapes of expandable elements depicted in the figures. For example, although some of the expandable elements described herein have substantially circular perimeters, and the perimeters expand as the expandable elements assume the hydrated state, it shall be understood that in other embodiments, the perimeters of the expandable elements can take on different shapes, such as substantially elliptical or triangular, for example. In addition, the invention encompasses embodiments in which the expandable elements expand further in one direction than in another. The invention also encompasses embodiments in which one or more expandable element is folded or rolled to reduce its profile in the dehydrated state. As such an expandable element expands, the expandable element automatically unfolds or unrolls.

The preceding specific embodiments are illustrative of the practice of the invention. It is to be understood, therefore, that other embodiments known to those skilled in the art or disclosed herein may be employed without departing from the invention or the scope of the claims. For example, the present invention further includes within its scope methods of making and using systems as described herein.

Many embodiments of the invention have been described. Various modifications may be made without departing from the scope of the claims. These and other embodiments are within the scope of the following claims.

1. A device comprising:
an elongate gastric band, having a radially expandable lumen, that forms a stoma opening in the stomach by encircling and partitioning a stomach into an upper stomach and a lower stomach; and
a hydrogel material positioned in the lumen that expands when hydrated to at least partially expand the lumen and reduce the size of the stoma opening.

2. The device of claim 1, wherein the hydrogel material comprises at least one of a single monolithic component, beads of hydrogel material, microspheric particles of hydrogel material, and a powdered hydrogel material.

3. The device of claim 1, wherein the hydrogel material comprises polyacrylonitrile and polyacrylamide copolymers.

4. The device of claim 1, where the hydrogel material is in a hydrated state.

5. The device of claim 4, wherein the hydrogel material is in a dehydrated state.

6. The device of claim 5, further including an injection port in fluid communication with the lumen that receives and delivers fluid to the dehydrated hydrogel material to at least partially hydrate the dehydrated hydrogel material.

7. The device of claim 6, wherein the fluid is delivered after the gastric band is implanted within a patient.

8. The device of claim 1, wherein the hydrogel material is in a partially hydrated state.

9. The device of claim 8, further including an injection port in fluid communication with the lumen that receives and delivers fluid to the partially hydrated hydrogel material to further hydrate the partially hydrated hydrogel material.

10. The device of claim 9, wherein the fluid is delivered after the gastric band is implanted within a patient.

11. The device of claim 1, wherein the gastric band has an inner diameter and an outer diameter when encircling the stomach, and wherein the inner diameter is controlled by at least one of a degree of hydration of the hydrogel material and an amount of hydrogel material positioned in the lumen.

12. The device of claim 1, wherein the gastric band further includes a first end, a second end, and a connection mechanism that connects the first end and the second end such that the gastric band encircles the stomach.

13. A device comprising:
a gastric occluding device positioned to restrict ingestion of food by a patient and having at least one expandable lumen;
an expandable material positioned within the lumen that expands when hydrated to at least partially expand the lumen.

14. The device of claim 13, wherein the expandable material is a hydrogel material.

15. The device of claim 14, wherein the hydrogel material comprises at least one of a single monolithic component, beads of hydrogel material, microspheric particles of hydrogel material, and a powdered hydrogel material.

16. The device of claim 13, wherein the hydrogel material comprises polyacrylonitrile and polyacrylamide copolymers.

17. The device of claim 13, where the hydrogel material is in a hydrated state.

18. The device of claim 17, wherein the hydrogel material is in a dehydrated state.

19. The device of claim 18, further including an injection port in fluid communication with the lumen that receives and delivers fluid to the dehydrated hydrogel material to at least partially hydrate the dehydrated hydrogel material.

20. The device of claim 19, wherein the fluid is delivered after the gastric band is implanted within a patient.

21. The device of claim 13, wherein the hydrogel material is in a partially hydrated state.

22. The device of claim 21, further including an injection port in fluid communication with the lumen that receives and delivers fluid to the partially hydrated hydrogel material to further hydrate the partially hydrated hydrogel material.

23. The device of claim 22, wherein the fluid is delivered after the gastric band is implanted within a patient.

24. A method comprising:
implanting a gastric band having at least one expandable lumen with material that expands when hydrated positioned therein such that the gastric band forms a stoma opening in the stomach of a patient by encircling and partitioning a stomach into an upper stomach and a lower stomach; and
injecting fluid into the expandable lumen to at least partially hydrate the material and to decrease the stoma opening.

25. The method of claim 24, wherein the material is dehydrated when implanted.

26. The method of claim 24, wherein the material is partially hydrated when implanted.

27. The method of claim 24, further including withdrawing fluid from the expandable lumen to adjust the stoma opening.

28. The method of claim 24, wherein injecting fluid into the expandable lumen comprises injecting fluid via a subcutaneous injection port in fluid connection with the expandable lumen.

29. A method comprising:
implanting a gastric band having at least one expandable lumen filled with a fully hydrated hydrogel material such that the gastric band forms a stoma opening in the stomach of a patient by encircling and partitioning a stomach into an upper stomach and a lower stomach.

30. A method comprising:
implanting a gastric band having at least one expandable lumen such that the gastric band forms a stoma opening in the stomach of a patient by encircling and partitioning a stomach into an upper stomach and a lower stomach; and
injecting a slurry of particulate hydrogel material into the expandable lumen to at least partially expand the lumen and decrease the stoma opening.

31. A device comprising:
an elongate gastric band having a radially expandable lumen that forms a stoma opening in the stomach by encircling and partitioning a stomach into an upper stomach and a lower stomach; and
an injection port in fluid connection with the lumen to receive and deliver a slurry of particulate hydrogel material into the expandable lumen to at least partially expand the lumen and decrease the stoma opening.
